(19) United States
(12) Patent Application Publication
(10) Pub. No.: US 2004/0247714 A1
(43) Pub. Date: Dec. 9, 2004

(54) Method and composition for enhancing vascular function

(76) Inventors: Suzanne Roe, Nuriootpa (AU); Darko Obradovic, Nuriootpa (AU); Kenneth Klingner, Nuriootpa (AU)

Correspondence Address: John E. Vandigriff, Suite 200, 190 N. Stemmons Frwy., Lewisville, TX, 75067, US

(21) Appl. No.: 10/340,463

(22) Filed: Jan. 13, 2003

(30) Foreign Application Priority Data
Oct. 23, 2002 (AU) 2002052212

(51) Int. Cl. A61K 35/78

(52) U.S. Cl. 424/766

(57) ABSTRACT

A method of improving vascular function comprising administering to a subject in need thereof grape seed extract. The grape seed extract is added to foodstuff products to form a functional food. The extract of the invention contains 30-90% w/w polyphenols measured as gallic acid equivalent. Polyphenols in grape seed extract may be present as oligomeric and polymeric proanthocyanidins. In a preferred form of the invention 60-90 % w/w of the total polyphenol component present is polymeric in form and more preferably has 2-16 monomeric units in each polymeric molecule. The polyphenols present in the composition of the invention contain less than 1% w/w flavonols.

### FIELD OF THE INVENTION

The present invention relates to a method of enhancing vascular function and to a composition for achieving the same. The composition of the present invention is derived from grape seed extract (GSE).

### BACKGROUND OF THE INVENTION

Polyphenol compounds obtained from plant sources have attracted attention because of their possible health benefits. The vine grape has become a commercial source of polyphenol because of the relative ease with which the material is available for extraction and the availability of significant volumes of the raw material for processing.

Vine products yield polyphenols from the grape skins, grape pulp and from grape seeds. The grape seed have been identified as having the highest concentrations of available polyphenols. Grape seed extracts (GSE) contain a high concentration of polyphenols, and most especially the proanthocyanidins. Included within the general group proanthocyanidins are the following compounds and their oligomers and polymers:

monomeric flavan-3-ols

(+)-catechin

(−)-epicatechin

Other flavan-3-ol entities which may be present in polymeric grape components include:

(+/−) gallocatechin

(−)-epicatechin 3-o-gallate

Other materials rich in polyphenols, notably Green Tea, which is especially rich in catechins, have been associated with antioxidant effects. Green Tea has, in epidemiological studies, been associated with protection from both heart disease and cancer.

Vascular function can be monitored by the measurement of flow-mediated dilation in blood vessels. Located at the interface between blood and a blood vessel wall the endothelium forms the lining of the blood vessel. The endothelium moderates many blood vessel functions and plays a critical role in the mechanics of blood flow and the regulation of coagulation. In a healthy artery, which also has a healthy endothelium, blood vessels are able to relax if the blood flow increases. This effect is called flow-mediated dilation (FMD).

Damage to the endothelium can lead to atherosclerotic plaques, the build-up of fatty materials within the walls of the arteries, and possibly to more serious cardiac conditions including angina and heart attacks.

It has been observed that patients who have impaired FMD are more likely to have coronary disease and also that those patients who have very impaired FMD are likely to have more events.

### SUMMARY OF THE INVENTION

Therefore, according to a first aspect of the present invention, although this need not be the broadest nor indeed the only aspect of the invention there is provided a method of improving vascular function comprising administering to a subject in need thereof grape seed extract.

It is suggested that an improvement in vascular function, as measured by changes in FMD relates to an improvement in overall cardiac health. Certainly those patients with coronary disease who have very impaired FMD have more events and subjects with impaired FMD are more likely to have coronary disease on angiography. It would therefore be logical to suppose that an improvement in the flow mediated dilation of vascular tissues would result in an improvement in overall cardiac health.

It can also be noted that drugs that have been shown to favourably influence outcome in coronary patients have been linked to improvements in FMD. The present invention is predicated on the observation that grape seed extract favourable influences FMD.

It is known that FMD and GTN dilatation is impaired in patients with moderate and severe coronary artery disease. Endothelial function as assessed by intracoronary acetylcholine predicts the rate of death and reinfarction in patients with coronary disease. FMD is related to coronary endothelial function and predicts the number of ischemic episodes suffered by patients with coronary artery disease and the late prognosis of patients with chest pain. Left ventricular mass is a predictor of outcome in hypertensive patients and is associated with impaired FMD. Accordingly, it can be suggested that an improvement in FMD following administration with GSE will ultimately be associated with an improvement in coronary health.

In one form of the invention, the grape seed extract is provided in the form of a powdered product. In alternative forms of the invention the grape seed extract is added to foodstuff products to form a functional food.

As an extract from a natural product that is a known food source GSE is likely to have high consumer acceptance. Further, it is possible to incorporate the GSE into food products in a manner that results in a final food product having attractive taste and flavour characteristics.

Some observers have also noted that FMD decreases after a high fat meal and is related to the triglyceride (TG). High TG levels after food ingestion are also considered a risk factor for coronary disease. In the case where GSE is administered within a food product it is thus possible that the improvement in FMD resulting from the ingestion of GSE may serve to counteract the negative effects of high fat foods.

Preferably, the grape seed extract is administered at a rate of 0.5-5 g/day. More preferably still said grape seed extract contains 30-90% w/w polyphenols measured as gallic acid equivalent

Polyphenols in grape seed extract may be present as oligomeric and polymeric proanthocyanidins. In a preferred form of the invention 60-90% w/w of the total polyphenol component present is polymeric in form and more preferably has 2-16 monomeric units in each polymeric molecule.

It should also be noted that the polyphenols present in the composition of the invention contain less than 1% w/w flavonols.

In accordance with a further aspect of the present invention there is provided a method of preparing a grape seed extract useful for improving vascular function and suitable for administration to a subject in need thereof, said method including the steps of:

separating grape seeds from grapes in a mechanical process;

subjecting said seeds to an extraction process in a suitable solvent to produce a crude extract; and

subjecting said crude extract to one or more purification steps.

Preferably, the one or more purification steps may further include filtration and evaporation of solvent.

### DESCRIPTION OF THE PREFERRED EMBODIMENT

1. Preparation of GSE Products

The GSE product of the invention may be prepared via a range of acceptable process conditions. The preparation of the compositions of the invention can be divided into two separate major processes

a) the preparation of the GSE; and

b) the preparation of a food product incorporating the GSE

Preparation of the GSE involves the separation of grapes seeds from the remaining grape material and the extraction of the polyphenolic material from the grape seeds themselves followed by subsequent processing steps that render the extract into a form suitable for further processing into food products.

Mechanical separation processes are used to remove the seeds from remaining grape material.

The extraction of the polyphenolic compounds of interest from the seed products may be achieved by any of a number of chemical extraction processes. For example, solvent extraction of polyphenolics into a suitable solvent such as ethanol and or water may be employed, followed by subsequent solvent removal by evaporation.

The extracted product may then be subjected to a spray drying or freeze-drying step to yield a powdered product. Alternatively, the product may be supplied in liquid form.

The seed product may be incorporated into food products. Typical food products that might incorporate the GSE formed in accordance with the invention include dairy foods such as yoghurt and other dairy products, cereals products including breads, biscuits and breakfast cereals; snack food products; fruit juices and other soft drinks, fruit products and confectionary. In addition to this the composition of the invention may be supplied as a more traditional form of supplement for example as a tablet or capsule or liquid tincture. A typical analysis of the composition is shown below:

| Test | Units | Range |
|---|---|---|
| Content of flavanol | % w/w measured as gailic acid | 30% to 90% |
| monomers, procyanidins | equivalents | |
| and proanthocyanidins | | |
| Content of polymeric | % w/w as a percent of total | 60% to 90% |
| procyanidins Degree | phenolics | 2 to 16 |
| of polymerisation | Average number of monomeric | |
| | units that make up the polymeric | |
| | molecules | |
| Content of Flavonols | % w/w | Less than 1 |

2. Effect of GSE on Vascular Function

Method

43 men and women with above average vascular risk from high cholesterol, smoking or high blood pressure were recruited by public advertisement and screened at the Clinical Research Unit, CSIRO Health Sciences and Nutrition in Adelaide. There were no exclusion criteria on the basis of medication or consumption of alcohol. Subjects were excluded if their BMI was >35 or if they suffered from diabetes mellitus, untreated metabolic disorders such as thyroid or adrenal disease, liver or kidney disease or had unstable coronary artery disease.

The trial was 12 weeks long and consisted of 3 four-weeks periods in a double-blind randomised crossover with control and active ingredients in 240 g of yoghurt. Active ingredients consisted of 2 g/day of grape seed extract (GSE) in the yoghurt. Blood samples and vascular compliance measures were taken at baseline and at the end of each period. The background diet was a low polyphenol, low quercetin diet. This was achieved by restricting tea and coffee to a maximum of 2 cups per day, restricting apples to one/day and forbidding red wine and onions throughout the 12 weeks. Flow mediated dilatation (FMD) was measured using ultrasound, vascular compliance using radial pulse analysis (HDI).

FMD was assessed in the brachial artery after blockage of blood flow in the forearm with a blood pressure cuff at 200 mm Hg for 5 minutes. The response of the vessel 5 minutes after 100 μg of glyceryl trinitrate sublingually was also assessed.

Brachial Artery Ultrasonography

Brachial artery ultrasonography was carried out in patients after a 12-hour fast and after resting supine for at least 15 minutes in a quiet, temperature controlled room (21 to 25° C.). Endothelium-dependent post-ischemic flow-mediated dilation (FMD) and endothelium-independent glyceryl trinitrate (GTN) mediated dilation (GTNMD) were measured During the ultrasound procedure, subjects rested supine in a quiet, temperature-controlled (24° C.) room. The left arm was supported comfortably in extension and supination. A high-resolution 12 mHz linear array transducer connected to an Acuson Aspen System (Acuson Pty Ltd., Mountain View, Calif., USA) was employed. Continuous EKG monitoring was performed in all studies. The transducer was placed 5 to 10 cm proximal to the antecubital crease and fixed in position by a stereotactic clamp. After good images were obtained, the edge-to-lumen interface was further optimized using depth and gain controls, and an edge enhancement function. Images were recorded continuously on s-VHS videotape (Sony MQSE 180) and 3 second clips were recorded on the ultrasound hard drive for retrospective analysis. A pneumatic tourniquet was placed around the left forearm, and after recording the baseline images for two minutes, the cuff was rapidly inflated to 200 mm Hg for five minutes. Forearm-reactive hyperemia was induced by sudden release of the cuff. Images were recorded continuously from 30 seconds before to 4 minutes after cuff release. A second resting scan was obtained at least 10 minutes after cuff deflation to ensure that the brachial artery diameter returned to the basal level. Two hundred micrograms of glyceryl trinitrate

(GTN) were administered sublingually and the images were recorded continuously for a further five minutes. Maximal FMD and GTNMD responses were calculated as % change in brachial artery diameter from baseline. The analytical (intra-observer) CV of the technique in our hands is in the order of 10%. The CV for repeated within-subject measurement was 15% (N=10) with a mean±standard deviation (SD) difference in FMD of 1.6±1.0%.

Statistical Analysis

Repeated-measures analysis of variance was calculated with type of yoghurt as the within-subject factor and with sex and order as the between subject factors. Where there was a significant treatment effect detected by repeated measures, paired students t tests were used to locate differences. Bivariate correlation was conducted using Pearson's correlation co-efficient. Analyses were performed with SPSS 10.0 for WINDOWS (SPSS Inc, Chicago). Significance was set at P<0.05

Results.

12 women and 24 men completed the study and one additional woman missed the last phase of treatment. Six subjects withdrew after commencement and 6 withdrew prior to commencement. All subjects missed 5 days of treatment when the study was temporarily suspended but it was considered that this was of little consequence in a 4 week treatment period.

Risk profile of subjects: 6 subjects had high blood pressure (5 on medication), 3 were smokers and 31 had high cholesterol (>5 mmol/L on finger prick). Two volunteers on atorvastatin to lower cholesterol stopped the medication prior to beginning the trial. The average cholesterol was 6.5 mmol/L (range 4.68 to 8.63), average age 58 years (range 34-70), weight 83.1 Kg (63.1 kg to 118.7 kg), BMI 28.4 (19.8-37.5). Mean blood pressure was 127 systolic and 74 diastolic.

10 Blood Pressure/Vascular Compliance (Table 3)

There was a weak (p<0.05) trend to a lowering of systolic blood pressure over the duration of the trial with a fall from 127 at baseline to 124 at week 12. This quite usual in clinical trials in which blood pressure is measured. There were no changes in any vascular parameter with treatment.

TABLE 3
Mean N = 35 complete measures
SD

| | Baseline | GSE | Control |
|---|---|---|---|
| Systolic BP (mmHg) | 127 ± 15 | 124 ± 14 | 124 ± 13 |
| Diastolic BP (mmHg) | 74 ± 9 | 73 ± 8 | 73 ± 9 |
| Mean BP (mmHg) | 94 ± 13 | 91 ± 18 | 91 ± 12 |
| Pulse pressure (mmHg) | 53 ± 9 | 51 ± 8 | 51 ± 8 |
| Pulse rate (beats/min) | 58 ± 8 | 59 ± 8 | 57 ± 7 |
| Estimated Cardiac Ejection | 335 ± 25 | 337 ± 24 | 333 ± 36 |
| Time (msec | | | |
| Estimated Stroke Volume (ml) | 93 ± 13 | 92 ± 12 | 94 ± 12 |
| Estimated Stroke volume | 47 ± 5 | 47 ± 4 | 47 ± 6 |
| Index (ml/m²) | | | |
| Estimated Cardiac output | 5.4 ± 0.7 | 5.5 ± 0.7 | 5.4 ± 0.7 |
| (L/min) | | | |
| Estimated Cardiac output | 2.8 ± 0.2 | 2.7 ± 0.3 | 2.7 ± 0.2 |
| Index (L/min/m²) | | | |
| Large artery elasticity index | 17.5 ± 4.6 | 18.4 ± 4.8 | 18.2 ± 4.7 |
| Small Artery Elasticity Index | 7.4 ± 3.8 | 7.4 ± 3.4 | 7.4 ± 3.2 |
| Systemic vascular resistance | 1364 ± 275 | 1345 ± 229 | 1352 ± 209 |
| Total Vascular Impedance | 131 ± 32 | 124 ± 33 | 127 ± 35 |

Flow Mediated Dilatation After Compression Release and GTN Dilatation.

Grape seed extract alone produced an absolute 1.1% greater dilatation compared with control (p<0.05). GTN induced dilatation was not influenced by GSE.

TABLE 4
Flow Mediated Dilation as measured by Ultrasound.
N = 35, Mean SD

| | Baseline | GSE | Control |
|---|---|---|---|
| Pre compression cm⁻² | 44.3 ± 6.3 | 45.1 ± 6.4 | 45.5 ± 7.3 |
| Post compression | 46.2 ± 5.8 | 47.4 ± 6.5 | 47.3 ± 7.3 |
| | (n = 30) | (n = 35) | (n = 36) |
| Change | 1.9¹² ± 1.3 | 2.3¹ ± 1.4 | 1.8² ± 1.3 |
| | (4.3%) | (5.1%) | (4.0%) |
| Pre GTN | 44.8 ± 7.1 | 45.8 ± 7.1 | 46.2 ± 7.1 |
| Post GTN | 52.1 ± 6.8 | 52.7 ± 6.9 | 52.8 ± 7.0 |
| | (n = 38) | (n = 30) | (n = 31) |
| Change | 7.3¹ ± 2.4 | 6.9¹² ± 2.3 | 6.5¹² ± 1.8 |
| | (16.3%) | (15.1%) | (14.1%) |

Treatments with different superscripts are different at p<0.05

The results confirm that the GSE of the invention and as prepared as described hereinabove favourably influences the endothelium enhancing nitric oxide production, release or slowing down oxidative destruction. The results also demonstrate that sufficient proanthocyanidins from GSE are absorbed to influence flow-mediated dilatation.

A range of other indicators of endothelial and vascular health were also tested and were not to be adversely affected by treatment with the composition of the invention.

The invention has been described by way of example. The examples are not, however, to be taken as limiting the scope of the invention in any way. Modifications and variations of the invention such as would be apparent to a skilled addressee are deemed to be within the scope of the invention.

1. A method of improving vascular function comprising administering to a subject in need thereof grape seed extract.

2. A method of improving cardiac health as measured by changes in measured flow mediated dilation comprising administering to a subject in need thereof grape seed extract.

3. A method of improving vascular function according to claim 1, comprising administering grape seed extract in the form of a powdered product.

4. A method of improving vascular function according to claim 1, comprising administering the grape seed extract in the form of a functional food.

5. A method according to claim 1, wherein the grape seed extract is administered at a rate of 0.5-5g/day.

6. A method according to claim 1, wherein said grape seed extract contains 30-90% w/w polyphenols measured as gallic acid equivalent.

7. A method according to claim 1, wherein 60-90% w/w of the total polyphenol component of said grape seed extract present is polymeric in form

8. A method according to claim 7, wherein said grape seed extract has 2-16 monomeric units in each polymeric molecule.

9. A method according to claim 1, wherein said grape seed extract contains less than 1% w/w flavonols.

10. A method of preparing a grape seed extract useful for improving vascular function and suitable for administration to a subject in need thereof, said method including the steps of:
separating grape seeds from grapes in a mechanical process;
subjecting said seeds to an extraction process in a suitable solvent to produce a crude extract; and
subjecting said crude extract to one or more purification steps.

11. A method according to claim 10, in which the one or more purification steps may further include filtration and evaporation of solvent.
